(19) United States
(12) Patent Application Publication
(10) Pub. No.: US 2022/0018976 A1
(43) Pub. Date: Jan. 20, 2022

(54) METHOD AND APPARATUS TO IMPROVE ROBUSTNESS IN A DIGITAL RADIOGRAPHIC CAPTURE DEVICE

(71) Applicant: CARESTREAM HEALTH, INC., Rochester, NY (US)

(72) Inventor: Todd D. BOGUMIL, Rochester, NY (US)

(21) Appl. No.: 17/296,335

(22) PCT Filed: Nov. 6, 2019
(86) PCT No.: PCT/US19/59956

§ 371 Date: May 24, 2021

Related U.S. Application Data
(60) Provisional application No. 62/781,159, filed on Dec. 18, 2018.

(51) Int. Cl. G01T 1/20; A61B 6/00

(52) U.S. Cl. CPC G01T 1/20189; A61B 6/4233; A61B 6/44; A61B 6/4283

(57) ABSTRACT

A digital radiographic detector having a core assembly and a housing enclosing the core assembly. Sidewalls of the housing have a thickness greater than the top and bottom sides of the housing. A first planar spring couples the core assembly to an interior surface of the housing. A second planar spring may be attached to the core assembly and abutting another interior surface of the housing.

### BACKGROUND OF THE INVENTION

The subject matter disclosed herein relates to digital radiographic (DR) detectors used with x-ray systems in medical imaging facilities.

Portable digital radiographic detectors have been widely deployed to improve diagnostic radiographic imaging productivity, image quality and ease of use. In particular, mobile or bedside radiographic imaging is conducted in locations such as intensive care units so that the patient does not need to be transported from their critical care environment. This type of imaging procedure is best served by a portable detector that is light weight and durable to improve ease of use and reliability.

Current digital radiographic detectors typically include an amorphous silicon TFT/photo diode image sensor array that is fabricated on glass using semiconductor processes that are similar to those used for flat panel displays. A scintillator is combined with the image sensor array along with required electronics for signal readout and processing onto an internal core plate which is contained within a durable housing to create the portable DR detector.

DR detectors may include elastic or cushion components, such as foam rubber or other materials, to protect the DR detector from impact and point load damage. Bumpers made from various protective materials placed between the core plate and the housing of a DR detector, as well as external bumpers at corners of the DR detector or bearing against other components, are prior art embodiments that can be improved.

### BRIEF DESCRIPTION OF THE INVENTION

Embodiments of the invention disclosed herein prevents damage to DR detector electronics housed within the DR detector when the DR cassette is subjected to durability tests such as drop shock, load and vibration. In some embodiments of the invention, planar and/or curved spring elements are bonded and/or brought to bear against an end cap and/or the DR detector housing to act as shock absorbers during impact. In one embodiment an additional structure above the core plate also serves to stiffen the structure to resist bending and point loading. Using embodiments of the invention disclosed herein, DR detectors passed quality tests involving drop shock at selected heights between about twelve (12) inches and thirty-six (36) inches drop heights, which were performed multiple times for each edge, face, and corner of the DR detectors without damage to the electronics housed therein. The described embodiments also passed a point load test.

A digital radiographic detector having a core assembly and a housing enclosing the core assembly. Sidewalls of the housing have a thickness greater than the top and bottom sides of the housing. A first planar spring couples the core assembly to an interior surface of the housing. A second planar spring may be attached to the core assembly and abutting another interior surface of the housing.

In one embodiment, a digital radiographic detector includes a core assembly and a five sided housing enclosing the core assembly. The five sided housing includes a top side, a bottom side and three sidewalls. The sidewalls are each thicker than the top and bottom sides. A spring is attached to the core assembly and is in physical contact against an inside surface of one of the side walls of the housing.

In another embodiment, a digital radiographic detector includes a core assembly and a four sided tubular housing enclosing the core assembly. The tubular housing has a rectangular cross section, a top side, a bottom side and sidewalls. The sidewalls are thicker than the top and bottom sides. Attachable and detachable end caps allow insertion of the core assembly into the tubular housing when one of the end caps is detached. A planar spring is attached to the core assembly and is in physical contact with an inside surface of at least one of the end caps or an inside surface of a sidewall.

In another embodiment, a digital radiographic detector includes a core assembly and a unitary housing enclosing the core assembly. The unitary housing having a top side, a bottom side and sidewalls. A spring member is fixed to the core assembly and is in physical contact with an inside surface of the housing to absorb a shock impacting an outside surface of the housing opposite the inside surface.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic perspective view of an exemplary x-ray system;

FIG. 2 is a schematic diagram of a photosensor array in a digital radiographic (DR) detector;

FIG. 3 is a perspective diagram of an exemplary DR detector;

FIG. 4 is a cross section diagram of an exemplary DR detector;

FIGS. 5A-5B are perspective views of exemplary core assembly components of a DR detector;

FIGS. 6A-6B are perspective views of additional exemplary board-side core assembly components of a DR detector;

FIGS. 7A-7B are perspective views of exemplary sensor-side core assembly components of a DR detector;

FIGS. 8A-8B are exploded perspective views of final DR detector assembly;

FIGS. 9A-9B are perspective views of completed DR detector assembly;

FIGS. 10A-10B are perspective views of exemplary support structures within the DR detector assembly;

FIGS. 11A-11B are perspective views of exemplary thermal dissipation structures within the DR detector assembly;

FIG. 12 is a cross section view of the thermal dissipation structures of FIGS. 11A-11B;

FIG. 13 is a top view of the DR detector core assembly;

FIG. 14A is a cross section view A-A of the DR detector core assembly of FIG. 13; and

FIG. 14B is a cross section view B-B of the DR detector core assembly of FIG. 13.

### DETAILED DESCRIPTION OF THE INVENTION

This application claims priority to U.S. Patent Application Ser. No. 62/781,159, filed Dec. 18, 2018, in the name of Todd D. Bogumil, and entitled METHOD AND APPARATUS TO IMPROVE ROBUSTNESS IN A DIGITAL RADIOGRAPHIC CAPTURE DEVICE, which is hereby incorporated by reference herein in its entirety.

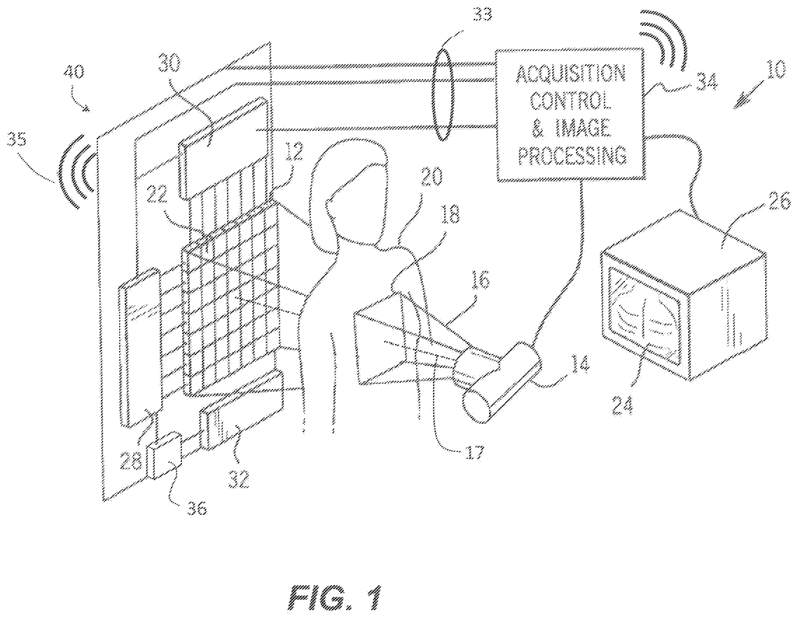

FIG. 1 is a perspective view of a digital radiographic (DR) imaging system 10 that may include a generally curved or planar DR detector 40 (shown in a planar embodiment and without a housing for clarity of description), an x-ray source 14 configured to generate radiographic energy (x-ray radiation), and a digital monitor, or electronic display, 26 configured to display images captured by the DR detector 40, according to one embodiment. The DR detector 40 may include a two dimensional array 12 of detector cells 22 (photosensors), arranged in electronically addressable rows and columns. The DR detector 40 may be positioned to receive x-rays 16 passing through a subject 20 during a radiographic energy exposure, or radiographic energy pulse, emitted by the x-ray source 14. As shown in FIG. 1, the radiographic imaging system 10 may use an x-ray source 14 that emits collimated x-rays 16, e.g. an x-ray beam, selectively aimed at and passing through a preselected region 18 of the subject 20. The x-ray beam 16 may be attenuated by varying degrees along its plurality of rays according to the internal structure of the subject 20, which attenuated rays are detected by the array 12 of photosensitive detector cells 22. The curved or planar DR detector 40 is positioned, as much as possible, in a perpendicular relation to a substantially central ray 17 of the plurality of rays 16 emitted by the x-ray source 14. In a curved array embodiment, the source 14 may be centrally positioned such that a larger percentage, or all, of the photosensitive detector cells are positioned perpendicular to incoming x-rays from the centrally positioned source 14. The array 12 of individual photosensitive cells (pixels) 22 may be electronically addressed (scanned) by their position according to column and row. As used herein, the terms “column” and “row” refer to the vertical and horizontal arrangement of the photo sensor cells 22 and, for clarity of description, it will be assumed that the rows extend horizontally and the columns extend vertically. However, the orientation of the columns and rows is arbitrary and does not limit the scope of any embodiments disclosed herein. Furthermore, the term “subject” may be illustrated as a human patient in the description of FIG. 1, however, a subject of a DR imaging system, as the term is used herein, may be a human, an animal, an inanimate object, or a portion thereof.

In one exemplary embodiment, the rows of photosensitive cells 22 may be scanned one or more at a time by electronic scanning circuit 28 so that the exposure data from the array 12 may be transmitted to electronic read-out circuit 30. Each photosensitive cell 22 may independently store a charge proportional to an intensity, or energy level, of the attenuated radiographic radiation, or x-rays, received and absorbed in the cell. Thus, each photosensitive cell, when read-out, provides information defining a pixel of a radiographic image 24, e.g. a brightness level or an amount of energy absorbed by the pixel, that may be digitally decoded by image processing electronics 34 and transmitted to be displayed by the digital monitor 26 for viewing by a user. An electronic bias circuit 32 is electrically connected to the two-dimensional detector array 12 to provide a bias voltage to each of the photosensitive cells 22.

Each of the bias circuit 32, the scanning circuit 28, and the read-out circuit 30, may communicate with an acquisition control and image processing unit 34 over a connected cable 33 (wired), or the DR detector 40 and the acquisition control and image processing unit 34 may be equipped with a wireless transmitter and receiver to transmit radiographic image data wirelessly 35 to the acquisition control and image processing unit 34. The acquisition control and image processing unit 34 may include a processor and electronic memory (not shown) to control operations of the DR detector 40 as described herein, including control of circuits 28, 30, and 32, for example, by use of programmed instructions, and to store and process image data. The acquisition control and image processing unit 34 may also be used to control activation of the x-ray source 14 during a radiographic exposure, controlling an x-ray tube electric current magnitude, and thus the fluence of x-rays in x-ray beam 16, and/or the x-ray tube voltage, and thus the energy level of the x-rays in x-ray beam 16. A portion or all of the acquisition control and image processing unit 34 functions may reside in the detector 40 in an on-board processing system 36 which may include a processor and electronic memory to control operations of the DR detector 40 as described herein, including control of circuits 28, 30, and 32, by use of programmed instructions, and to store and process image data similar to the functions of standalone acquisition control and image processing system 34. The image processing system may perform image acquisition and image disposition functions as described herein. The image processing system 36 may control image transmission and image processing and image correction on board the detector 40 based on instructions or other commands transmitted from the acquisition control and image processing unit 34, and transmit corrected digital image data therefrom. Alternatively, acquisition control and image processing unit 34 may receive raw image data from the detector 40 and process the image data and store it, or it may store raw unprocessed image data in local memory, or in remotely accessible memory.

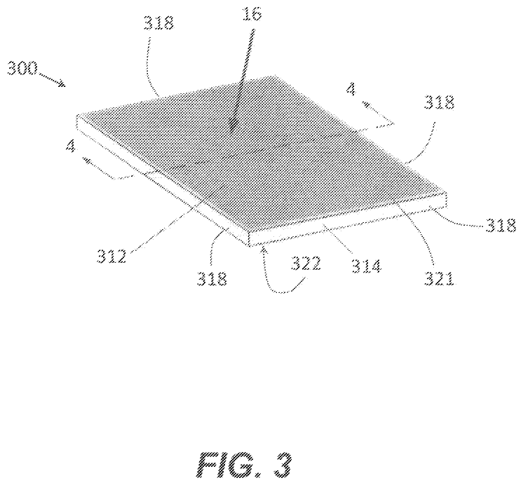

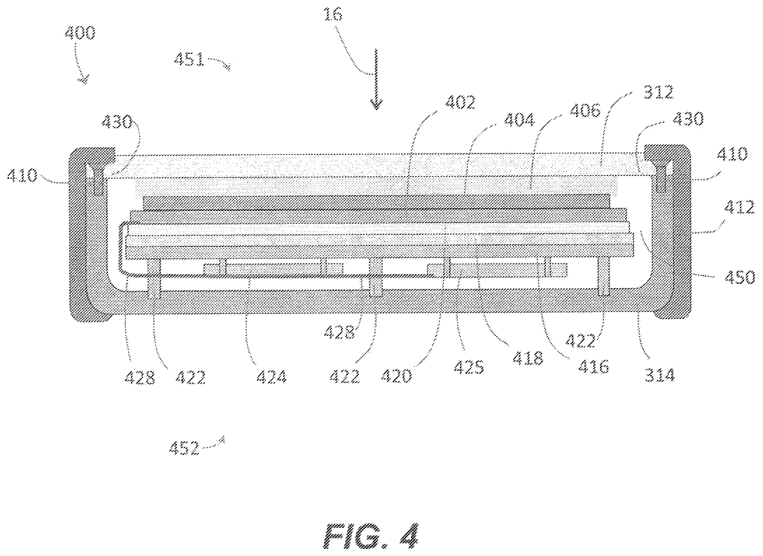

With regard to a direct detection embodiment of DR detector 40, the photosensitive cells 22 may each include a sensing element sensitive to x-rays, i.e. it absorbs x-rays and generates an amount of charge carriers in proportion to a magnitude of the absorbed x-ray energy. A switching element may be configured to be selectively activated to read out the charge level of a corresponding x-ray sensing element. With regard to an indirect detection embodiment of DR detector 40, photosensitive cells 22 may each include a sensing element sensitive to light rays in the visible spectrum, i.e. it absorbs light rays and generates an amount of charge carriers in proportion to a magnitude of the absorbed light energy, and a switching element that is selectively activated to read the charge level of the corresponding sensing element. A scintillator, or wavelength converter, may be disposed over the light sensitive sensing elements to convert incident x-ray radiographic energy to visible light energy. Thus, in the embodiments disclosed herein, it should be noted that the DR detector 40 (or DR detector 300 in FIG. 3 or DR detector 400 in FIG. 4) may include an indirect or direct type of DR detector.

Examples of sensing elements used in sensing array 12 include various types of photoelectric conversion devices (e.g., photosensors) such as photodiodes (P-N or PIN diodes), photo-capacitors (MIS), photo-transistors or photoconductors. Examples of switching elements used for signal read-out include a-Si TFTs, oxide TFTs, MOS transistors, bipolar transistors and other p-n junction components.

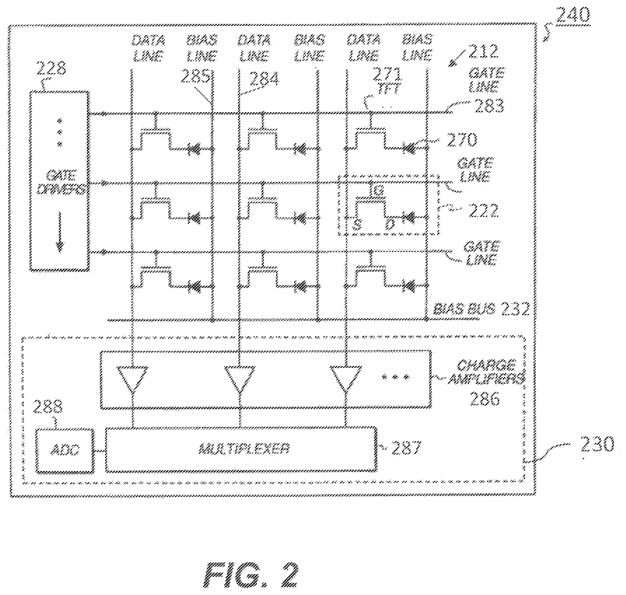

FIG. 2 is a schematic diagram 240 of a portion of a two-dimensional array 12 for a DR detector 40. The array of photosensor cells 212, whose operation may be consistent with the photosensor array 12 described above, may include a number of hydrogenated amorphous silicon (a-Si:H) n-i-p photodiodes 270 and thin film transistors (TFTs) 271 formed as field effect transistors (FETs) each having gate (G), source (S), and drain (D) terminals. In embodiments of DR detector 40 disclosed herein, such as a multilayer DR detector (400 of FIG. 4), the two-dimensional array of photosensor cells 12 may be formed in a device layer that abuts adjacent layers of the DR detector structure, which adjacent layers may include a rigid glass layer or a flexible polyimide layer or a layer including carbon fiber without any adjacent rigid layers. A plurality of gate driver circuits 228 may be electrically connected to a plurality of gate lines 283 which control a voltage applied to the gates of TFTs 271, a plurality of readout circuits 230 may be electrically connected to data lines 284, and a plurality of bias lines 285 may be electrically connected to a bias line bus or a variable bias reference voltage line 232 which controls a voltage applied to the photodiodes 270. Charge amplifiers 286 may be electrically connected to the data lines 284 to receive signals therefrom. Outputs from the charge amplifiers 286 may be electrically connected to a multiplexer 287, such as an analog multiplexer, then to an analog-to-digital converter (ADC) 288, or they may be directly connected to the ADC, to stream out the digital radiographic image data at desired rates. In one embodiment, the schematic diagram of FIG. 2 may represent a portion of a DR detector 40 such as an a-Si:H based indirect flat panel, curved panel, or flexible panel imager.

Incident x-rays, or x-ray photons, 16 are converted to optical photons, or light rays, by a scintillator, which light rays are subsequently converted to electron-hole pairs, or charges, upon impacting the a-Si:H n-i-p photodiodes 270. In one embodiment, an exemplary detector cell 222, which may be equivalently referred to herein as a pixel, may include a photodiode 270 having its anode electrically connected to a bias line 285 and its cathode electrically connected to the drain (D) of TFT 271. The bias reference voltage line 232 can control a bias voltage of the photodiodes 270 at each of the detector cells 222. The charge capacity of each of the photodiodes 270 is a function of its bias voltage and its capacitance. In general, a reverse bias voltage, e.g. a negative voltage, may be applied to the bias lines 285 to create an electric field (and hence a depletion region) across the pn junction of each of the photodiodes 270 to enhance its collection efficiency for the charges generated by incident light rays. The image signal represented by the array of photosensor cells 212 may be integrated by the photodiodes while their associated TFTs 271 are held in a non-conducting (off) state, for example, by maintaining the gate lines 283 at a negative voltage via the gate driver circuits 228. The photosensor cell array 212 may be read out by sequentially switching rows of the TFTs 271 to a conducting (on) state by means of the gate driver circuits 228. When a row of the pixels 22 is switched to a conducting state, for example by applying a positive voltage to the corresponding gate line 283, collected charge from the photodiode in those pixels may be transferred along data lines 284 and integrated by the external charge amplifier circuits 286. The row may then be switched back to a non-conducting state, and the process is repeated for each row until the entire array of photosensor cells 212 has been read out. The integrated signal outputs are transferred from the external charge amplifiers 286 to an analog-to-digital converter (ADC) 288 using a parallel-to-serial converter, such as multiplexer 287, which together comprise read-out circuit 230.

This digital image information may be subsequently processed by image processing system 34 to yield a digital image which may then be digitally stored and immediately displayed on monitor 26, or it may be displayed at a later time by accessing the digital electronic memory containing the stored image. The flat panel DR detector 40 having an imaging array as described with reference to FIG. 2 is capable of both single-shot (e.g., static, radiographic) and continuous (e.g., fluoroscopic) image acquisition.

FIG. 3 shows a perspective view of an exemplary prior art generally rectangular, planar, portable wireless DR detector 300 according to an embodiment of DR detector 40 disclosed herein. The DR detector 300 may include a flexible substrate to allow the DR detector to capture radiographic images in a curved orientation. The flexible substrate may be fabricated in a permanent curved orientation, or it may remain flexible throughout its life to provide an adjustable curvature in two or three dimensions, as desired. The DR detector 300 may include a similarly flexible housing portion 314 that surrounds a multilayer structure, or core assembly, comprising a flexible photosensor array portion 22 of the DR detector 300. The housing portion 314 of the DR detector 300 may include a continuous, rigid or flexible, x-ray opaque material or, as used synonymously herein a radio-opaque material, surrounding an interior volume of the DR detector 300. The housing portion 314 may include four flexible edges 318, extending between the top side 321 and the bottom side 322, and arranged substantially orthogonally in relation to the top and bottom sides 321, 322. The bottom side 322 may be continuous with the four edges and disposed opposite the top side 321 of the DR detector 300. The top side 321 comprises a top cover 312 attached to the housing portion 314 which, together with the housing portion 314, substantially encloses the core assembly in the interior volume of the DR detector 300. The top cover 312 may be attached to the housing 314 to form a seal therebetween, and be made of a material that passes x-rays 16 without significant attenuation thereof, i.e., an x-ray transmissive material or, as used synonymously herein, a radiolucent material, such as a carbon fiber, carbon fiber embedded plastic, polymeric, elastomeric and other plastic based material.

With reference to FIG. 4, there is illustrated in schematic form an exemplary cross-section view along section 4-4 of the exemplary embodiment of the DR detector 300 (FIG. 3). For spatial reference purposes, one major surface, or side, of the DR detector 400 may be referred to as the top side 451 and a second major surface, or side, of the DR detector 400 may be referred to as the bottom side 452, as used herein. The core assembly layers, or sheets, may be disposed within the interior volume 450 enclosed by the housing 314 and top cover 312 and may include a flexible curved or planar scintillator layer 404 over a curved or planar the two-dimensional imaging sensor array 12 shown schematically as the device layer 402, which may also be referred to hereinbelow as the sensor. The scintillator layer 404 may be directly under (e.g., directly connected to) the substantially planar top cover 312, and the imaging array 402, or sensor, may be directly under the scintillator 404. Alternatively, a flexible layer 406 may be positioned between the scintillator layer 404 and the top cover 312 as part of the core assembly layered structure to allow adjustable curvature of the core assembly layered structure and/or to provide shock absorption. The flexible layer 406 may be selected to provide an amount of flexible support for both the top cover 312 and the scintillator 404, and may comprise a foam rubber type of material. The layers just described comprising the core assembly layered structure each may generally be formed in a rectangular shape and defined by edges arranged orthogonally and disposed in parallel with an interior side of the edges 318 of the housing 314, as described in reference to FIG. 3.

A substrate layer 420 may be disposed under the imaging array 402, such as a rigid glass layer, in one embodiment, or flexible substrate comprising polyimide or carbon fiber upon which the array of photosensors 402 may be formed to allow adjustable curvature of the array, and may comprise another layer of the core assembly layered structure. Under the substrate layer 420 a radio-opaque shield layer 418, such as lead, may be used as an x-ray blocking layer to help prevent scattering of x-rays passing through the substrate layer 420 as well as to block x-rays reflected from other surfaces in the interior volume 450. Readout electronics, including the scanning circuit 28, the read-out circuit 30, the bias circuit 32, and processing system 36 (all shown in FIG. 1) may be formed adjacent the imaging array 402 or, as shown, may be disposed below frame support member 416 in the form of integrated circuits (ICs) electrically connected to printed circuit boards (PCBs) 424, 425. The imaging array 402 may be electrically connected to the readout electronics 424 (ICs) over a flexible connector 428 which may comprise a plurality of flexible, sealed conductors known as chip-on-film (CoF) connectors.

X-ray flux may pass through the radiolucent top panel cover 312, in the direction represented by an exemplary x-ray beam 16, and impinge upon scintillator 404 where stimulation by the high-energy x-rays 16, or photons, causes the scintillator 404 to emit lower energy photons as visible light rays which are then received in the photosensors of imaging array 402. The frame support member 416 may connect the core assembly layered structure to the housing 314 and may further operate as a shock absorber by disposing elastic pads (not shown) between the frame support beams 422 and the housing 314. Fasteners 410 may be used to attach the top cover 312 to the housing 314 and create a seal therebetween in the region 430 where they come into contact. In one embodiment, an external bumper 412 may be attached along the edges 318 of the DR detector 400 to provide additional shock-absorption.

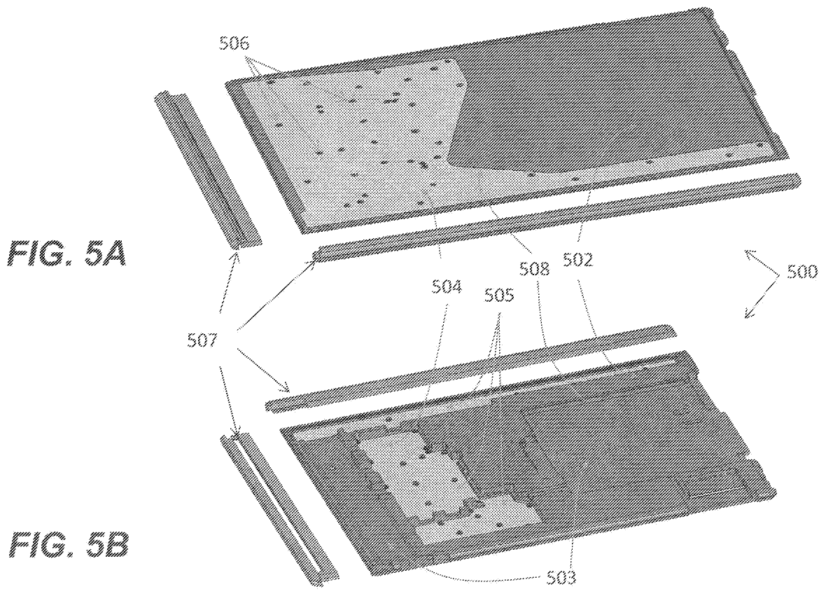

Referring to FIGS. 5A and 5B, there is illustrated one embodiment of a DR detector wherein a multi layered core assembly 500 includes a substantially planar high density foam layer 502 machined to form recessed pockets 503 on at least one two major side thereof. A plate 504 formed from a metal, such as aluminum, is positioned in a recessed pocket on a top side of the foam layer 502 as shown in FIG. 5A. The metal plate, or ground plane, 504 may be glued to the foam layer 502 to secure it in position or it may be secured in position by one or more sidewalls 508 of the recessed pocket. Recessed pockets 503 are also machined in a bottom side of the foam layer 502 as shown in FIG. 5B, which bottom side pockets 503 will have printed circuit boards (PCBs) or electronic components placed therein. The foam layer 502 is also machined to form cutouts 505 therethrough wherein PCBs and other electronics may be placed therein from the bottom side and positioned against the ground plane 504 which may be placed therein from the top side as shown in FIG. 5A, and as described herein. The ground plane 504 functions as an electrical ground for the electronic components to be assembled as described herein. As shown in FIG. 5B, the metal ground plane 504 is visible through the cutouts 505 before placement therein of PCBs or other electronic components. In one embodiment, the high density foam 502 may be formed by molding it into the shape having cutouts 505 and recessed pockets 503 as shown, such as by injection molding.

The metal ground plane 504 includes a plurality of holes 506, some of which may be threaded, for attaching electrical and mechanical components. Protective end caps 507, also made from the same or similar high density foam as the foam layer 502 are positioned along the edges of the foam layer 502 after electronic components are positioned thereon. As referred to herein, a width dimension of the multi layered core assembly 500 is parallel to the shorter sides thereof as compared to the length dimension which is parallel to the longer sides of the multi layered core assembly 500. The top and bottom sides of the multi layered core assembly 500, as shown in FIGS. 5A and 5B, respectively, together with further detector assembly layers as described herein may be referred to as major surfaces of the multi layered core assembly 500. As shown in FIG. 5A, an area of the top side major surface of the multi layered core assembly 500 made from the foam layer 502 may be about the same or greater than an area made from the metal ground plane 504. According to embodiments of the multi layered core assembly 500 disclosed herein, an area of the metal ground plane 504 may be designed to cover from about 40% of the top side major surface area up to about 65% of the top side major surface area. The foam used for foam layer 502 and the end caps 507, and other foam components described herein may include high density, thermoplastic, closed cell foams having good heat and flame resistance, heat and electrical insulating properties, a high strength to weight ratio and low moisture absorption. A high density foam such as a polyetherimide based thermoplastic foam or a poly vinylidene fluoride based foam may be used. Alternatively, the foam components may be formed from silicone or rubber.

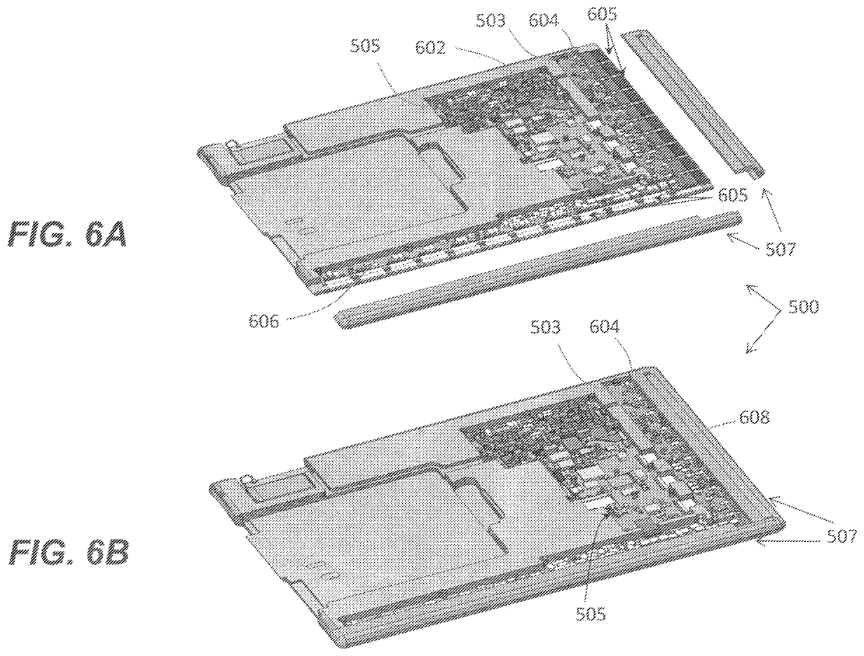

FIGS. 6A and 6B illustrate the bottom side of the multi layered core assembly 500 (rotated 180° compared to the view of FIG. 5B) having PCBs placed in the cutouts 505 and in at least one recessed pocket 503. The PCBs 602, 606, 608, placed in the cutouts 505 abut the grounding plane 504 and may be connected thereto using screws through the PCB into the holes 506 of the grounding plane 504. Electrically conductive screws may be used to electrically connect the PCBs to the grounding plane 504 and/or the PCBs and ground plane may be separately electrically connected together. The PCB 604 is positioned in the recessed pocket 503. The PCBs may include, for example, a power distribution electronics PCB 602, a PCB 604 containing read out integrated circuits (ROICs), a PCB 606 for gate driver circuitry, and a PCB 608 having a main processor section. Some of the PCBs 606 having the gate driver circuitry 606 and/or the PCBs 604 with ROICs may include conductive communication lines (CoFs) 605 extending from the PCBS 604, 606, around an edge of the foam layer 502 and ground plane 504 assembly to the top side of the multi layered core assembly 500 to enable digital communication between the PCB electronics and the radiographic sensor array on the top side of the multi layered core assembly 500 which includes the two-dimensional array of photo-sensitive cells, as described herein. As shown in FIG. 6B, the protective foam ends caps 507 may be positioned on the edges of the foam layer 502 and ground plane 504 assembly over the CoFs 605.

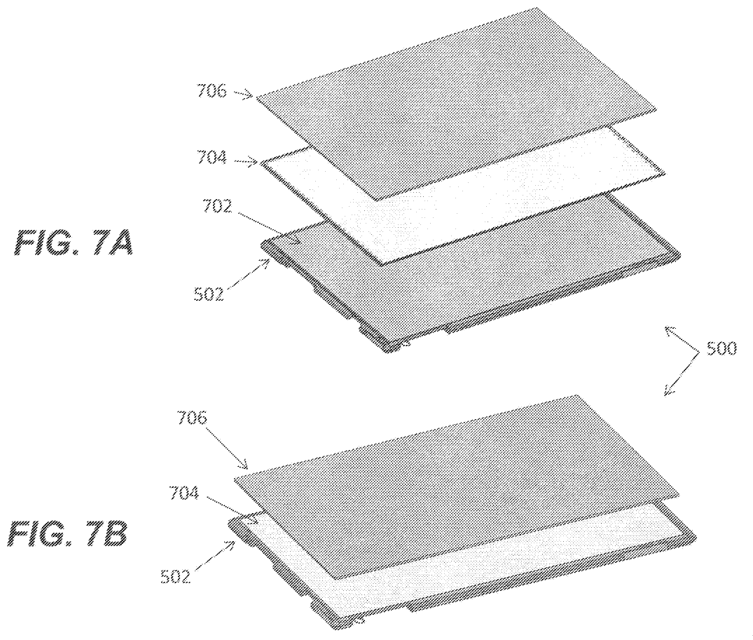

FIGS. 7A-7B illustrate the top side of the multi layered core assembly 500. A lead layer 702 is positioned against the top side of the multi layered core assembly 500 to provide shielding against x-rays that may scatter near the DR detector assembly. The lead layer 702 has an area substantially equivalent to an area of a major surface of the multi layered core assembly 500 and, in the embodiments described herein, is the only metal layer in the multi layered core assembly 500 having as extensive an area as the multi layered core assembly 500 itself. The metal grounding plane 504 may cover about 65% of the area covered by the lead layer 702, as mentioned herein. A sensor layer 704 which may comprise a scintillator layer laminated onto the two-dimensional array of photosensitive cells, is placed on the lead layer 702 and is seated on the top side of the multi layered core assembly 500 as shown in FIG. 7B. The sensor layer 704 may further include a substrate upon which the two-dimensional array of photosensors is formed. The substrate may include a rigid glass substrate or it may be formed as a flexible substrate such as a polyimide substrate. A shock absorbing foam layer 706 is positioned on top of the sensor layer 704 and typically abuts an inside surface of an enclosure (housing) for the multi layered core assembly 500. Altogether, the multi layered core assembly 500 may have a thickness of between about one-eighth inch and about one-half inch including the PCB circuitry attached thereto.

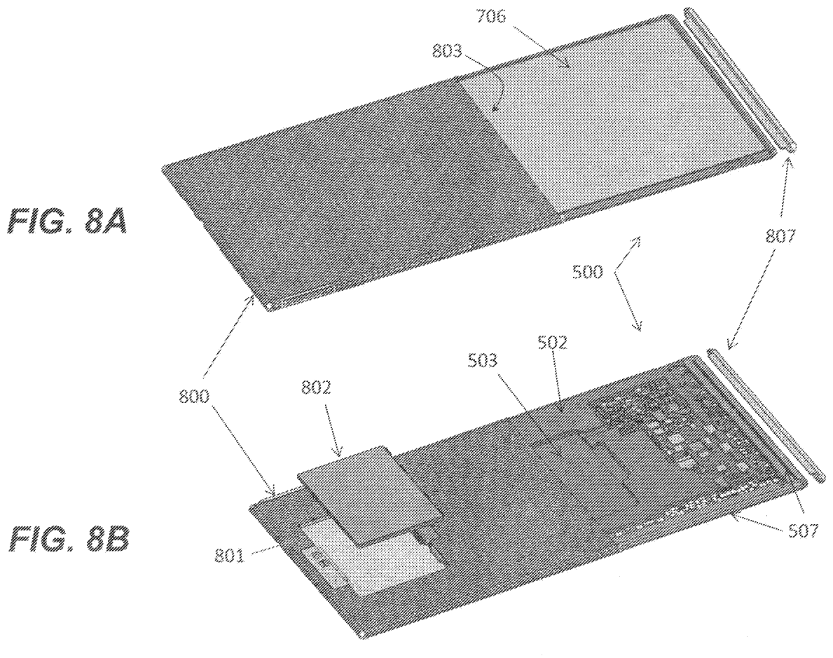

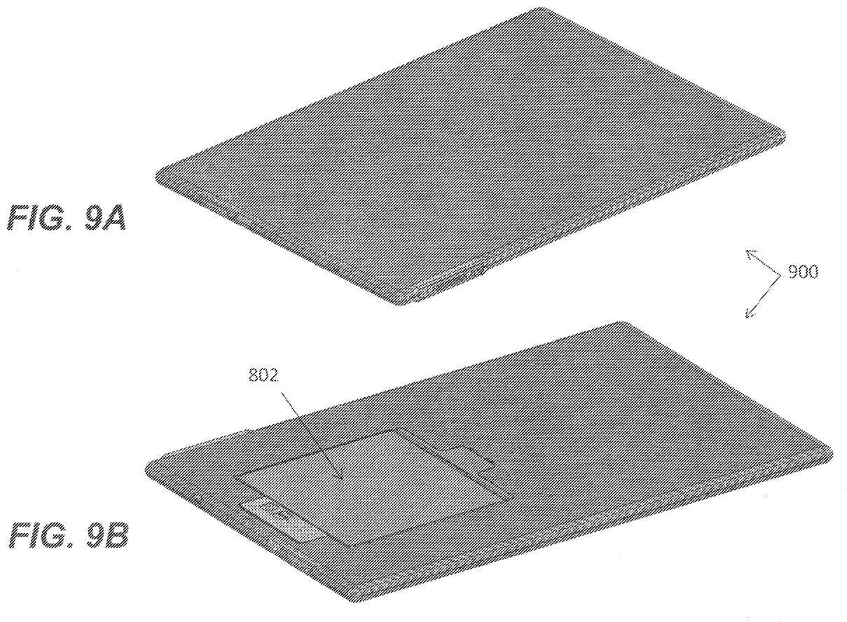

FIGS. 8A-8B illustrate the top and bottom sides, respectively, of the multi layered core assembly 500, as assembled, being inserted into an open end 803 of an enclosure, or housing, 800 which enclosure 800 may also be referred to as having corresponding top and bottom sides. A bottom side of the enclosure 800, as shown in FIG. 8B, includes an opening 801 for a battery 802 to be placed therethrough into a corresponding recessed pocket 503 of the foam layer 502 after the multi layered core assembly 500 is fully inserted into the enclosure 800. Subsequently, an enclosure end cap 807, or cover, may be positioned in the open end 803 of the enclosure 800 to seal the open end 803 of the enclosure 800 to completely enclose the core assembly 500 and complete the assembly of the DR detector 900 (FIG. 9). Such an end cap 807 may be formed out of aluminum and positioned in thermal contact with one or more of the PCBs, as described herein. The open end 803 may have a height of between about one-eighth inch and about one-half inch, similar to the thickness of the multi layered core assembly 500 to allow slidable entry of the multi layered core assembly 500 through the open end 803. In one embodiment, the shock absorbing foam layer 706 may be compressed to half its thickness upon the multi-layered core assembly 500 being inserted into the enclosure 800. The enclosure 800, as shown, may be a carbon fiber based material such as a twill type of carbon fiber, however, other carbon fiber types of enclosures may be used such as carbon fiber embedded plastics. In addition to carbon fiber, magnesium, aluminum, and plastic enclosures may be used, similar in form as the carbon fiber enclosure 800.

As shown, the enclosure 800 is a five-sided enclosure formed as a unitary integrated whole having only one open end parallel to a width of the multi-layer core assembly 500. In another separate embodiment, the enclosure 800 may be formed as a four-sided enclosure, such as a flat tube having a rectangular cross section with two opposing open ends. In such an embodiment, the multi-layer core assembly 500 could be inserted into either open end of the four-sided enclosure and two enclosure end caps 807 could be used to seal the opposing open ends of such an enclosure. FIGS. 9A-9B illustrate the top and bottom sides, respectively, of a completed assembly of the DR detector 900, wherein the battery 802 may be removed and replaced through the bottom side of the DR detector 900 as described herein.

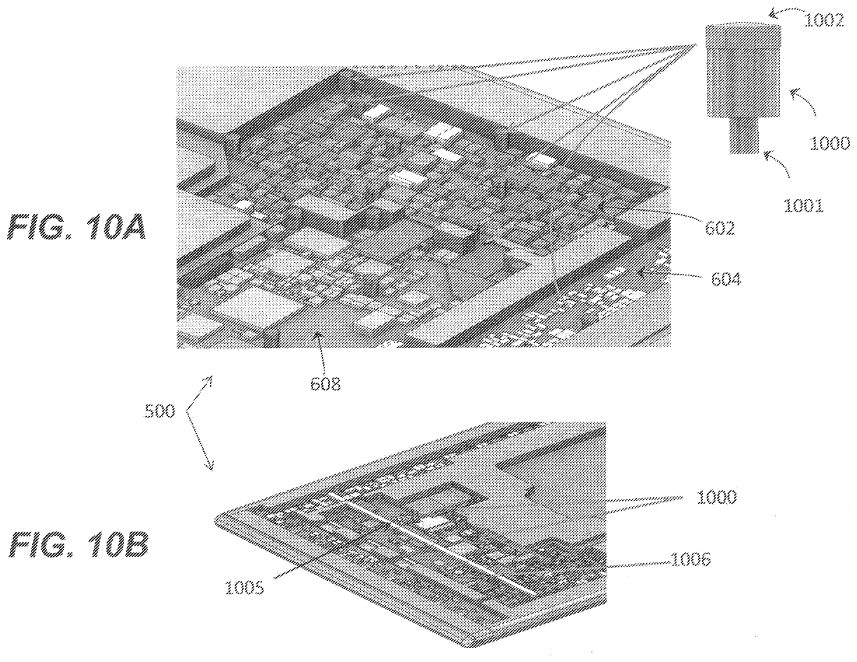

FIGS. 10A (close-up) and 10B illustrate the bottom side of the multi layered core assembly 500 (rotated about 180° in the two figures) having PCBs placed in the cutouts 505 and in at least one recessed pocket 503. A deflection limiter 1000 is used to attach the PCBs 602, 604, 608, to the grounding plane 504. The deflection limiter 1000 may include a bottom portion 1001 that may be inserted through a hole in the PCBs 602, 604, 608, into the holes 506 of the grounding plane 504 to secure the PCBs 602, 604, 608, directly to the grounding plane 504. In one embodiment, the bottom portion 1001 of the deflection limiter 1000 may be threaded to engage a threaded hole 506 of the grounding plane 504 to screw the PCBs 602, 604, 608, against the grounding plane 504. In one embodiment, the deflection limiter 1000 may be made entirely from a conductive material to electrically connect the PCBs 602, 604, 608, to the grounding plane 504. In addition, the deflection limiters 1000 may be disposed in locations selected to prevent excessive deflection of the enclosure 800 by providing a pillar to contact an interior surface of the enclosure 800 when the multi-layer core assembly 500 is inserted therein and so support the enclosure 800 to prevent excessive deflection thereof. An upper surface 1002 of the deflection limiter 1000 may be formed in a convex (domed) shape to prevent edges of the deflection limiter from marring an interior surface of the enclosure 800 coming into contact with the deflection limiter 800. Another feature of the multi layered core assembly 500 used to strengthen rigidity of the DR detector assembly is a carbon fiber stiffening beam 1005 positioned along a width dimension of the multi layered core assembly 500. The carbon fiber stiffening beam 1005 may be attached to the PCBs using one or more brackets 1006 or they may be attached to the tops of selectively positioned deflection limiters 1000.

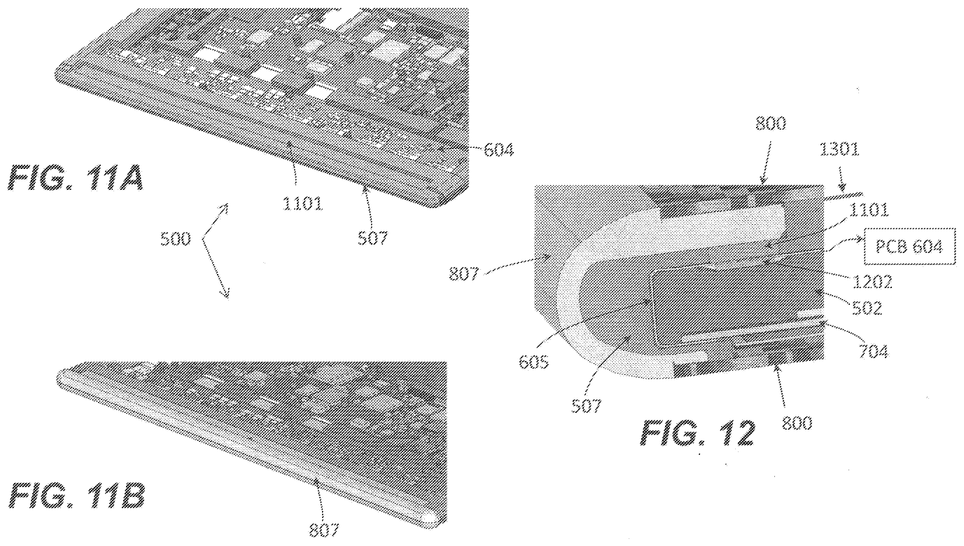

FIGS. 11A and 11B illustrate the bottom side of the multi layered core assembly 500 having a thermally conductive pad 1101 formed in the protective foam end cap 507 that is adjacent the PCB 604 containing the ROICs described herein. FIG. 11B shows the aluminum enclosure cap 807 in position on the protective foam end cap 507 without the enclosure 800 for illustration purposes. The thermally conductive pad 1101 may be used to provide thermal dissipation of heat generated by electronics in the multi layered core assembly 500. Preferably, the thermally conductive pad 1101 is in thermal contact with the aluminum enclosure cap 807 placed on the protective foam end cap 507, as shown in FIG. 12. FIG. 12 is a close-up cross section of an edge of the multi layered core assembly 500 as shown in FIG. 11B, which edge is parallel to the width of the multi-layer core assembly 500. With reference to FIG. 12, the thermally conductive pad 1101 is adjacent to and in thermal contact with an IC chip 1202 of the CoF 605. The CoF 605 extends around an edge of the foam layer 502, as described herein, and is electrically connected to the sensor layer 704 at one end, and is electrically connected to the ROICs of PCB 604 at another end. The IC chip 1202 of the CoF 605 may be a source of heat generation that, without a thermal exit pathway to an external environment of the DR detector 900, may cause a malfunction of the CoF 605 electronics, for example. Thus, the thermally conductive pad 1101 provides a portion of a thermal exit pathway by physically contacting the IC chip 1202 and absorbing heat therefrom. When the external aluminum enclosure cap 807 is in position to cover the open end of the enclosure 800, as shown, the aluminum enclosure cap 807 physically contacts the thermally conductive pad 1101 to absorb heat therefrom and functions as another portion of a thermally conductive exit pathway to dissipate heat from the thermally conductive pad 1101 to the external environment.

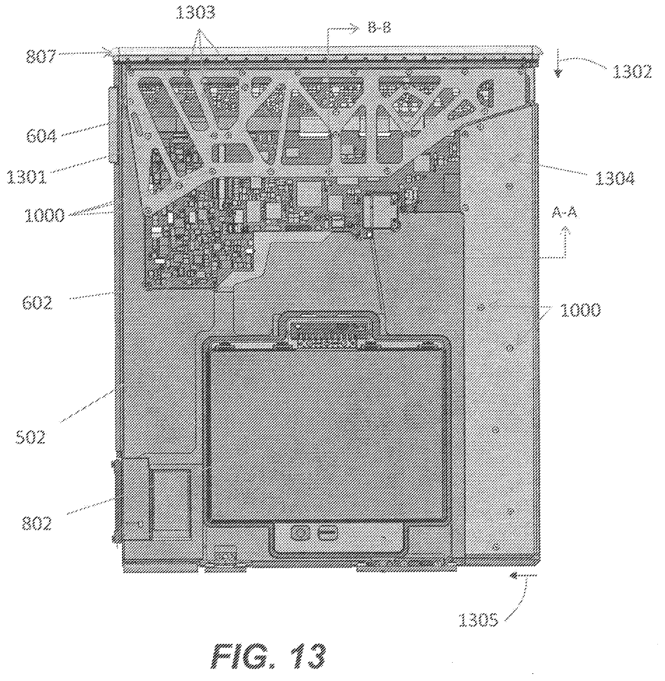

FIG. 13 illustrates a bottom side of the multi layered core assembly 500 in one embodiment, with housing 800 removed, wherein a thin, planar, ribbed compression spring element 1301, which may be made from carbon fiber, may be positioned over PCBs 602, 604, to provide flexible compliance in the downward direction 1302 from an impact on the end cap 807. The planar, ribbed compression spring element 1301 may be attached at its lower edge to two or more deflection limiters 1000. The end cap 807 may be rigidly attached to the housing or enclosure (FIG. 12). The planar spring 1301 may be positioned to abut the end cap 507 (FIG. 12) or it may be bonded thereto to provide a flexible compliance to a certain degree to reduce shock traveling from an impact to the end cap 807 into the sensor 704, for example. The thin ribs 1303 of the planar spring member 1301 bend and contort to distribute shock load from an impact at the end cap 807.

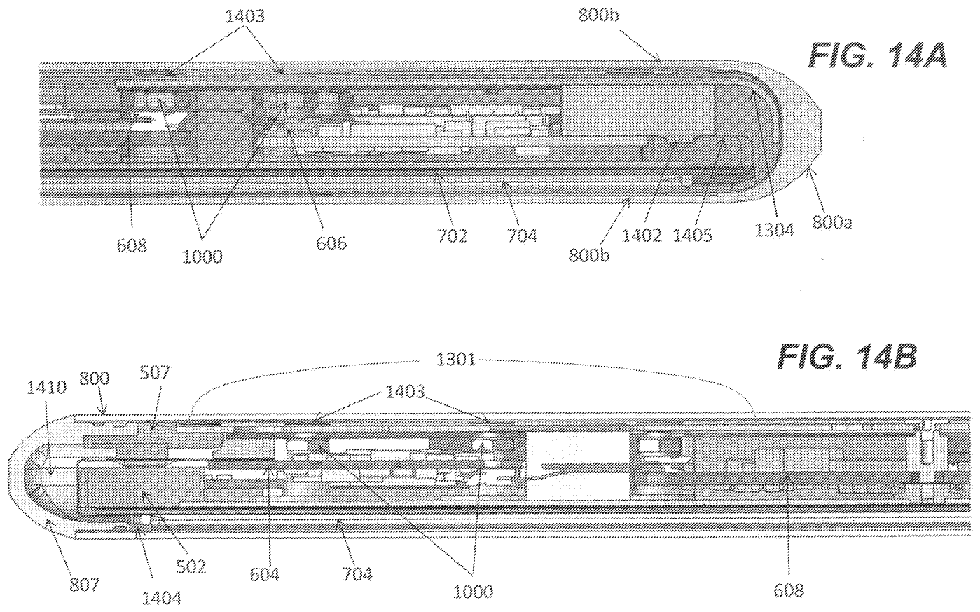

A second planar spring element 1304 covers the gate driver PCB 606 and may be attached thereto using the deflection limiters 1000. Spring element 1304 may bear against the inside of the housing 800. A flexible compliance or cushioning may be provided by the second spring element 1304 in a side-to-side direction 1305 because of a curvature (FIG. 14A) of the spring element 1304. The curved spring element 1304 may facilitate installation of the multi layered core assembly 500 by sliding the core assembly 500 into the housing 800. The curved spring may be configured to bear against an inside surface of the multi layered core assembly 500 or it may be spaced therefrom by a gap, as it serves to constrain movement of the core assembly 500. In one embodiment, it may be bolted to the top of the deflection limiters and curves upward (toward the top side of the multi layered core assembly 500) to make contact with the interior side walls of the enclosure 800.

FIG. 14A is a cross-section view A-A of FIG. 13 that shows the curved spring element 1304 bearing against a side wall 800a of the housing 800 for side impact 1305 shock absorption. An IC chip 1402 of the gate driver side CoF 1405 operates in a similar fashion as the IC chip 1202 of ROIC side CoF 605 of FIG. 12. Other components visible in FIG. 14A described previously include sensor/scintillator layers 704 and lead layer 702. Screws 1403 may be used to secure the curved spring element 1304 to the deflection limiters 1000, in a similar fashion as the planar, ribbed compression spring element 1301 is fastened to its deflection limiters 1000 (FIG. 13). The carbon fiber housing 800, or enclosure, gradually increases in thickness in the side wall 800a (up to between about 2 to 8 mm) to safely absorb a side impact 1305. The shape of the housing operates as an impact absorbing system together with the spring elements 1301, 1304, described herein. Impacts coming from the right 1305, as seen in FIG. 14A may compress the side wall and be absorbed by the housing. The impact may compress the side wall which is rigid enough to absorb shock and deflect the impact load without damage. The thin upper and lower surfaces (fascia) of the housing 800b bow out to absorb the impact. Inside the housing, the curved spring element 1304 also deflects to absorb impact shock.

FIG. 14B is a cross-section view B-B of FIG. 13 that shows an additional x-section through planar, ribbed compression spring element 1301. FIG. 14B shows an alternate embodiment of the enclosure end cap 807 wherein the protective end cap 507 is removed from a space 1410 (compare FIG. 12) to allow the multi layered core assembly 500 to slide within the housing 800 a small amount to absorb shock. There may be an intentional gap 1404 between the sensor layers 704 and enclosure end cap 807 so that a side impact allows the core assembly to slide within the housing to absorb shock. The compression spring element 1301 may be fastened or bonded to the protective end cap 507 for retention.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A digital radiographic detector comprising:
a core assembly;
a housing having five sides and enclosing the core assembly but for an open sixth side of the housing, the housing having a top side, a bottom side and three sidewalls therebetween, the housing integrally formed with the top side and the bottom side, the sidewalls each having a thickness greater than that of the top side and the bottom sides; and
a curved spring attached to the core assembly and in physical contact against an inside surface of one of the side walls of the housing.

2. The digital radiographic detector of claim 1, wherein the housing sidewalls are thicker in a middle portion equidistant from the top and bottom sides than in a portion immediately adjacent to either of the top and bottom sides.

3. The digital radiographic detector of claim 1, further comprising an attachable and detachable end cap to allow insertion of the core assembly into the five-sided housing when the end cap is detached therefrom, and to completely enclose the inserted core assembly in the housing when the end cap is attached to the open sixth side of the housing.

4. The digital radiographic detector of claim 3, wherein the end cap is detachable from the sixth side of the housing to allow slidably removing the core assembly through the open sixth side of the housing.

5. The digital radiographic detector of claim 3, further comprising a thermally conductive element in contact with the core assembly and in contact with the end cap.

6. The digital radiographic detector of claim 1, wherein the curved spring comprises a major surface substantially parallel to the top surface of the housing and a flex portion that curves approximately ninety degrees away from a plane of the major surface, the flex portion flexibly abutting the inside surface of the sidewall of the housing to provide absorption of an impact against the outside surface of the sidewall.

7. A digital radiographic detector comprising:
a core assembly;
a four sided tubular housing enclosing the core assembly, the tubular housing having a rectangular cross section, a top side, a bottom side and sidewalls therebetween, the housing integrally formed with, the top side and the bottom side, the sidewalls having a thickness greater than that of the top side and the bottom side;
attachable and detachable end caps to allow insertion of the core assembly into the tubular housing when at least one of the end caps is detached therefrom; and
a planar spring attached to the core assembly and in physical contact with at least one of the end caps.

8. The digital radiographic detector of claim 7, wherein the housing sidewalls are thicker in a middle portion equidistant from the top and bottom sides than in a portion immediately adjacent to either of the top and bottom sides.

9. The digital radiographic detector of claim 7, wherein the detachable end caps completely enclose the inserted core assembly in the housing when the end caps are attached to opposite ends of the tubular housing.

10. The digital radiographic detector of claim 9, further comprising a thermally conductive element in contact with the core assembly and in contact with at least one of the end caps, wherein said at least one of the end caps is made from a thermally conductive metal.

11. The digital radiographic detector of claim 7, further comprising a curved spring element fastened to the core assembly and flexibly abutting an inside surface of a sidewall of the housing.

12. The digital radiographic detector of claim 11, wherein the curved spring member comprises a major surface substantially parallel to the top surface of the detector and a flex portion that curves approximately ninety degrees away from a plane of the major surface, the flex portion flexibly abutting the inside surface of the sidewall of the housing to provide shock absorption of an impact against the outside surface of the sidewall.

13. A digital radiographic detector comprising:
a core assembly;
a unitary housing enclosing the core assembly, the housing having a top side, a bottom side and sidewalls therebetween, the sidewalls having a thickness greater than that of the top side and the bottom side; and
a spring member fixed to the core assembly and in physical contact with an inside surface of the housing to absorb a shock impacting an outside surface of the housing opposite the inside surface.

14. The digital radiographic detector of claim 13, wherein the housing sidewalls are thicker in a middle portion equidistant from the top and bottom sides than in a portion immediately adjacent to either of the top and bottom sides.

15. The digital radiographic detector of claim 13, further comprising a detachable cover to enclose an open end of the housing and to completely enclose the core assembly within the housing when the cover is attached thereto.

16. The digital radiographic detector of claim 15, further comprising a thermally conductive element in contact with the core assembly and in contact with the cover when the cover is attached to the housing, wherein said cover is made from a thermally conductive metal.

17. The digital radiographic detector of claim 13, wherein the spring member abuts an inside surface of a sidewall of the housing.

18. The digital radiographic detector of claim 15, wherein the spring member abuts an inside surface of the cover.

19. The digital radiographic detector of claim 17, wherein the spring member comprises a major surface substantially parallel to the top surface of the housing and a flex portion that curves approximately ninety degrees away from a plane of the major surface, the flex portion flexibly abutting the inside surface of the sidewall to provide shock absorption of an impact against the outside surface of the sidewall.
